(19) United States
(12) Patent Application Publication
(10) Pub. No.: US 2023/0218850 A1
(43) Pub. Date: Jul. 13, 2023

(54) OXYGEN INHALATION NASAL PRONG DEVICE

(71) Applicant: Shenyang RMS Medical Tech Co., Ltd, Liaoning (CN)

(72) Inventors: Chengguang ZHOU, Liaoning (CN); Shuang LI, Liaoning (CN); Kuncheng GAI, Liaoning (CN); Pan GAO, Liaoning (CN)

(73) Assignee: Shenyang RMS Medical Tech Co., Ltd, Liaoning (CN)

(21) Appl. No.: 18/176,789

(22) Filed: Mar. 1, 2023

Related U.S. Application Data
(63) Continuation of application No. PCT/CN2021/121082, filed on Sep. 27, 2021.

(30) Foreign Application Priority Data
Oct. 14, 2020 (CN) 202022289107.6

(51) Int. Cl. A61M 16/06

(52) U.S. Cl. CPC A61M 16/0672; A61M 16/0683

(57) ABSTRACT

An oxygen inhalation nasal prong device, including a catheter assembly, a nasal prong assembly, a lanyard assembly and a neck strap assembly, where the catheter assembly is a hollow tubular structure; the nasal prong assembly is arranged at one end of the catheter assembly, the other end of the catheter assembly is configured to communicate with an oxygen supply device, and the nasal prong assembly is connected with the catheter assembly in a through connection way; the lanyard assembly is arranged on the nasal prong assembly, and the lanyard assembly is configured to be sleeved on a head of a patient, so that the nasal prong assembly is fixed at a nostril of the patient; the neck strap assembly is configured to fix the other end of the catheter assembly to neck of the patient.

### CROSS-REFERENCE TO RELATED APPLICATIONS

This application is a continuation of International Application No. PCT/CN2021/121082, filed on Sep. 27, 2021, which claims priority to Chinese Patent Application No. 202022289107.6, filed on Oct. 14, 2020. The disclosures of the aforementioned applications are hereby incorporated by reference in their entireties.

### TECHNICAL FIELD

The present application relates to the field of medical devices, in particular to an oxygen inhalation nasal prong device.

### BACKGROUND

In clinical medicine, oxygen delivery is one of the most basic nursing tasks, and especially for a patient undergoing a respiratory therapy, the oxygen delivery is often needed and use of an oxygen inhalation nasal prong is required during such a process. Oxygen inhalation nasal prong is a device used for the patient to inhale oxygen in medical treatment, one end thereof is connected with an oxygen supply source and the other end thereof is inserted into the patient's nose. With the development of science and technology, there are more and more kinds of oxygen inhalation nasal prongs, and their functions are becoming more and more powerful.

At present, the oxygen inhalation nasal prong is simple in structure, convenient to wear and widely used. However, under the condition of long-term wearing, the air outlet of the nasal prong extends into the patient's nasal cavity, which will cause side effects such as nasal cavity deformation. Moreover, due to the internal structure of the nose and the need to breathe out through the nose, condensed water is easily formed and accumulated at the position of the nasal prong, which will easily choke into the patient's nasal cavity, causing uncomfortable of the patient and sometimes leading to nasal bacterial infection, and thus resulting in health hazards. In addition, the existing oxygen inhalation nasal prong is also very easy to fall out, causing the prong to fall out from the patient's nasal cavity, and affecting the nasal comfort.

Therefore, there is an urgent need to develop an oxygen inhalation nasal prong device, which can not only prevent condensed water from easily accumulating at the nasal prong position so as to deliver oxygen to patients comfortably to protect life and health of the patients, but also avoid the problem that the oxygen inhalation nasal prong device is easy to fall out and displace.

### SUMMARY

The present application provides an oxygen inhalation nasal prong device, which is used to solve at least the technical problems that condensed water is easy to be accumulated at a nasal prong position, which affects the comfort of oxygen delivery, and that the oxygen inhalation nasal prong device is easy to fall out and displace.

In order to achieve the above object, the present application provides an oxygen inhalation nasal prong device, including a catheter assembly, a nasal prong assembly, a lanyard assembly and a neck strap assembly, where the catheter assembly is a hollow tubular structure; the nasal prong assembly is arranged at one end of the catheter assembly, and the other end of the catheter assembly is configured to communicate with an oxygen supply device, and the nasal prong assembly is connected with the catheter assembly in a through connection way; the lanyard assembly is arranged on the nasal prong assembly, and the lanyard assembly is configured to be sleeved on a head of a patient, so that the nasal prong assembly is fixed at a nostril of the patient; the neck strap assembly is configured to fix the other end of the catheter assembly to neck of the patient.

According to the oxygen inhalation nasal prong device provided by the present application, the nasal prong assembly is fixed at the nostril of the patient through the lanyard assembly, so that the nasal prong assembly is prevented from easily falling off from the nostril of the patient and thus affecting the use effect; in addition, the neck strap assembly is arranged, so that a section of the catheter assembly far away from the nasal prong assembly is fixed at the neck of the patient, and the problems that the catheter assembly is easy to move due to an integral gravity and thus the nasal prong assembly partially moves down or flip over and falls out are reduced. The present application can effectively reduce the phenomenon that the oxygen inhalation nasal prong device is easy to fall out and displace, and protect the life and health of the patient.

In a possible embodiment, the nasal prong assembly includes a nasal prong body and a pair of nostril protruding pillars connected to a side of the nasal prong body, the nasal prong body is internally provided with a contracted airflow cavity, and the pair of nostril protruding pillars are each internally provided with an air outlet through-connected with the airflow cavity.

In a possible embodiment, an inner wall of one side of the airflow cavity has an inclined surface, there is an inclination angle θ between the inclined surface and a plane of a nostril end of the patient, and an inner wall of the other side of the airflow cavity has an arc surface.

In a possible embodiment, the inclination angle θ remains unchanged along an inclined direction of the inclined surface relative to the plane.

In a possible embodiment, the inclined surface is provided at one side of the airflow cavity close to the air outlet through-communicated with the airflow cavity, and the arc surface is provided at the other side of the airflow cavity far away from the air outlet through-communicated with the airflow cavity.

In a possible embodiment, the arc surface expands away from the inclined surface when it approaches an air inlet end of the airflow cavity; and the arc surface gradually approaches the inclined surface when it moves away from the air inlet end of the airflow cavity until it intersects with the inclined surface.

In a possible embodiment, the inclination angle θ of the inclined surface is 5°-15°.

In a possible embodiment, the inclination angle θ of the inclined surface is 50°.

In the oxygen inhalation nasal prong device provided by the present application, the contracted airflow cavity conforms to the gas flowing law, has a small airflow resistance, and is not easy to form condensed water; and the inclined surface of the airflow cavity has a certain inclination angle, so that the condensed water can flow back into the catheter assembly, to prevent the condensed water from accumulating at the nasal prong position and choking into the patient's nasal cavity.

In a possible embodiment, the lanyard assembly includes a first lanyard connecting member and a second lanyard connecting member, which are respectively arranged at both sides of the nasal prong body, and the first lanyard connecting member and the second lanyard connecting member are integrally formed with the nasal prong body;

the lanyard assembly further includes a lanyard connected between the first lanyard connecting member and the second lanyard connecting member.

In a possible embodiment, the lanyard is provided with an adjusting buckle.

In a possible embodiment, the lanyard repeatedly passes through the adjusting buckle to adjust a length of the lanyard.

In a possible embodiment, the catheter assembly includes an air conducting tube, an upper joint and a lower joint, where the upper joint and the lower joint are respectively connected at both ends of the air conducting tube, and the nasal prong body is connected with the upper joint in a through connection way.

In a possible embodiment, the air conducting tube is provided with a lanyard clamp.

In a possible embodiment, the air conducting tube is a threaded tube.

In a possible embodiment, the oxygen inhalation nasal prong device further includes a joint assembly, the joint assembly is through-connected to the other end of the catheter assembly, and the joint assembly includes a machine joint and a joint connecting member connected to one end of the machine joint, and the joint connecting member is connected to the lower joint.

In the oxygen inhalation nasal prong device provided by the present application, the first lanyard connecting member and the second lanyard connecting member are integrally formed with the nasal prong body, so that when the oxygen inhalation nasal prong device is worn, the first lanyard connecting member and the second lanyard connecting member are more fit closely to the patient's face after the nasal prong body is placed at a philtrum position of the patient, and have a small pressure on the patient's face, so that a potential damage to skin of a patient with sensitive skin is reduced, and the device is comfortable to wear.

In addition to the technical problems solved by the embodiments of the present application, the technical features constituting the technical solution and the beneficial effects brought by the technical features of these technical solutions as described above, other technical problems solved by the wearable oxygen inhalation nasal prong device provided by the embodiment of the present application, other technical features included in the technical solution and the beneficial effects brought by these technical features will be further explained in detail in the specific embodiments.

### BRIEF DESCRIPTION OF DRAWINGS

In order to more clearly explain the technical solutions in the embodiments of the present application or in the prior art, the drawings needed to be used in the descriptions of the embodiments or the prior art will be briefly introduced below. Obviously, the drawings in the following descriptions are some embodiments of the present application, and for ordinary people in the field, other drawings can be obtained according to these drawings without paying creative labor.

FIG. 1 is a front side view of an oxygen inhalation nasal prong device provided by an embodiment of the present application.

FIG. 2 is a right side view of the oxygen inhalation nasal prong device provided by an embodiment of the present application.

FIG. 3 is a schematic diagram of a three-dimensional structure of the oxygen inhalation nasal prong device provided by an embodiment of the present application.

FIG. 4 is a sectional view of the oxygen inhalation nasal prong device provided by an embodiment of the present application.

FIG. 5 is a partial structural sectional view of the oxygen inhalation nasal prong device provided by an embodiment of the present application.

FIG. 6 is a sectional view of a joint assembly of the oxygen inhalation nasal prong device provided by an embodiment of the present application.

FIG. 7 is a sectional view of a nasal prong assembly of the oxygen inhalation nasal prong device provided by an embodiment of the present application.

### DESCRIPTION OF REFERENCE SYMBOLS

10—Catheter assembly;
11—Air conducting tube;
111—Airflow channel;
12—Lanyard clamp;
13—Upper joint;
14—Lower joint;
141—Groove;
20—Nasal prong assembly;
21—Nasal prong body;
211—Airflow cavity;
212—Arc surface;
213—Inclined surface;
214 Air inlet end;
22—Nostril protruding pillar;
221—Air outlet;
30—Lanyard assembly;
31—First lanyard connecting member;
32—Lanyard;
33—Connecting buckle;
34—Second lanyard connecting member;
35—Adjusting buckle;
40—Neck strap assembly;
41—Neck strap;
42—Spring buckle;
43—Neck strap buckle;
50—Joint assembly;
51—Machine joint;
52—Joint connecting member; and
521—Claw.

### DESCRIPTION OF EMBODIMENTS

In order to make the purpose, technical solution and advantages of the present application more clear, the technical solution in the present application will be described clearly and completely with reference to the drawings of the present application. Obviously, the described embodiments are part of the embodiments of the present application, but not all of them. Based on the embodiments in the present application, all other embodiments obtained by those with ordinary skill in this field without creative work belong to the protection scope of the present application.

Clinically, by supplying oxygen to a patient, a hypoxia condition caused by various reasons can be rectified, thereby promoting the metabolism of patients' body tissues and maintaining life activities. At present, there are several commonly used interfaces for the patients in respiratory therapy, including tracheal intubation, mouth mask and nasal oxygen tube, where the oxygen inhalation nasal prong device, as one kind of nasal oxygen tube, is simple in structure, convenient to wear, and thus is widely used.

In use, the oxygen inhalation nasal prong device is connected with the oxygen supply device through a machine, and oxygen is delivered to a nasal cavity of the patient through an air conducting tube, so as to relieve various clinical symptoms caused by lack of oxygen. However, the existing oxygen inhalation nasal prong device is easy to fall out and displace, which is partly caused by self-weight of the device, especially when the patient turns over and accidentally hits the device. In addition, the existing oxygen inhalation nasal prong device still has the problem that condensed water is easy to accumulate in the nasal prong, which causes the patient to be choked due to the condensed water inhaled when breathing, and even causes bacterial infection in the patient's nose, affecting the patient's life and health.

In order to solve the problems that the existing oxygen inhalation nasal prong device is easy to fall out and displace, and that the patient is easy to be choked due to the condensed water inhaled when breathing, the present application improves the structure of the oxygen inhalation nasal prong device and provides an oxygen inhalation nasal prong device.

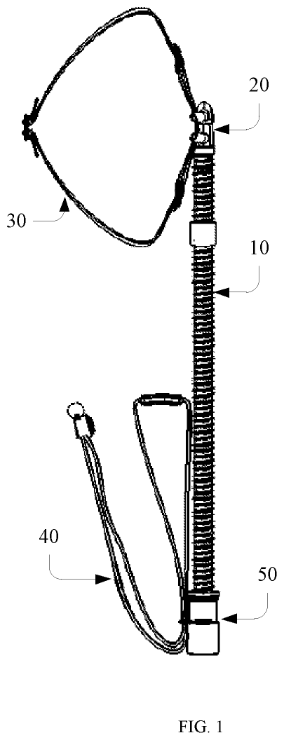

Hereinafter, the oxygen inhalation nasal prong device provided by embodiments of the present application will be described with reference to FIGS. 1-7.

In view of the above background, referring to FIG. 1, the present application provides an oxygen inhalation nasal prong device, which includes a catheter assembly 10, a nasal prong assembly 20, a lanyard assembly 30 and a neck strap assembly 40, where the catheter assembly 10 is a hollow tubular structure.

Specifically, the “hollow” means that the catheter assembly 10 has a channel running through both ends of the catheter assembly 10, and the channel provides a flow path for delivery of oxygen.

Referring to FIG. 1, a nasal prong assembly 20 is arranged at one end of a catheter assembly 10, and the other end of the catheter assembly is configured to communicate with an oxygen supply device, and the nasal prong assembly 20 is connected with the catheter assembly 10 in a through connection way, and oxygen provided by the oxygen supply device is delivered to the nasal prong assembly 20 through the catheter assembly 10.

The lanyard assembly 30 is arranged on the nasal prong assembly 20, and the lanyard assembly 30 is configured to be sleeved on head of a patient, so that the nasal prong assembly 20 is fixed at a nostril of the patient, which improves the fixation stability and prevents the nasal prong assembly 20 from falling off or displace due to actions such as turning over, accidental collision of the patient, etc., thereby providing life safety guarantee for the patient.

The neck strap assembly 40 is configured to fix the other end of the catheter assembly 10 to the neck of the patient (for example, being sleeved on a neck of a patient), and the neck strap assembly 40 is configured to fix a section of the catheter assembly 10 far away from the nasal prong assembly 20, so as to avoid the catheter assembly 10 to fall down due to gravity and increase the stability of use.

The oxygen inhalation nasal prong device provided by the present application is used for direct delivery of oxygen or delivery of wetted oxygen between an oxygen source and the patient during oxygen inhalation, and has a simple and reasonable structure, is convenient to use, comfortable to wear and is not easy to fall off.

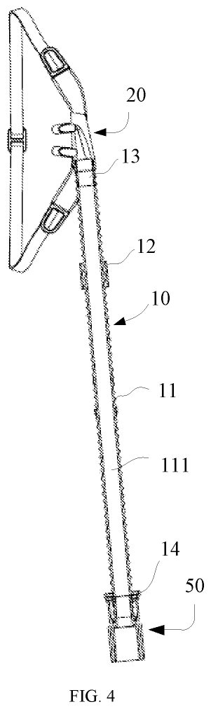

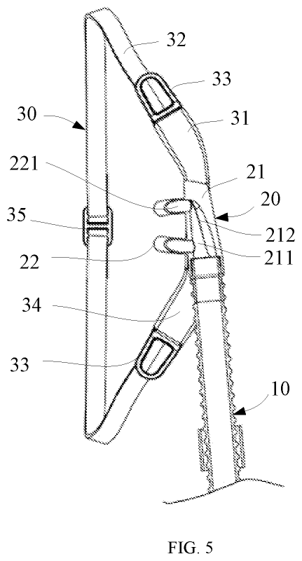

Referring to FIGS. 4 and 5, the nasal prong assembly 20 includes a nasal prong body 21 and a pair of nostril protruding pillars 22 connected to a side of the nasal prong body 21, that is, the number of the nostril protruding pillars 22 is two. Of course, in some examples, the number of the nostril protruding pillars 22 includes, but is not limited to, one pair, or can be more than two, so that the remaining nostril protruding pillar(s) 22 can be used as backup, and when one of the nostril protruding pillars 22 fails, the nostril protruding pillar(s) 22 as backup can be used.

Specifically, the nasal prong body 21 and a pair of nostril protruding pillars 22 are integrally formed into monolithic structure, thereby ensuring good air tightness.

Referring to FIG. 5, the nasal prong body 21 has a contracted airflow cavity 211. The contracted airflow cavity 211 means that a diameter of the airflow cavity 211 is gradually reduced along an airflow direction, and specifically, the diameter of the airflow cavity 211 is gradually reduced in the direction close to the nostril protruding pillar 22. The pair of nostril protruding pillars 22 are each provided with an air outlet 221 through-connected with the airflow cavity 211. When in use, each of the pair of nostril protruding pillars 22 is each inserted into the nasal cavity of the patient, and oxygen entering the airflow cavity 211 is output by the air outlet 221. The structure of the air outlet 221 conforms to the nasal cavity structure of the ordinary people, which reduces the possibility of damage to the nasal cavity due to long-term wearing.

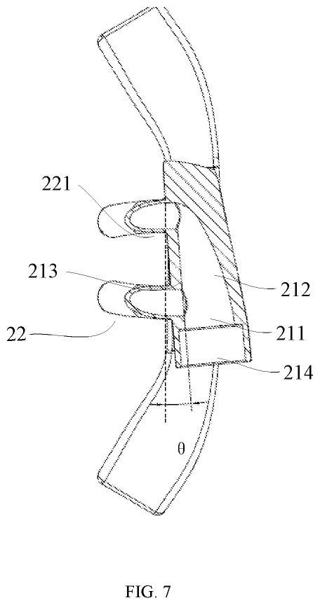

Referring to FIGS. 5 and 7, an inner wall of one side of the airflow cavity 211 close to the air outlet 221 has an inclined surface 213, and the inner wall of the other side of the airflow cavity 211 far away from the air outlet 221 has an arc surface 212. Specifically, the arc surface 212 is an arc curved surface with smooth transition.

The reason why the airflow cavity 211 exhibits a contracted shape is that the arc surface 212 expands away from the inclined surface 213 when it approaches an air inlet end 214 of the airflow cavity 211; and the arc surface 212 gradually approaches the inclined surface 213 when it moves away from the air inlet end 214 of the airflow cavity 211 until it intersects with the inclined surface 213.

In one possible embodiment, there is an inclination angle θ between the inclined surface 213 and a plane of a nostril end of the patient.

In one possible embodiment, the inclination angle θ remains unchanged along an inclined direction of the inclined surface 213 relative to the plane.

In one possible embodiment, the inclination angle θ of the inclined surface 213 is 5°-15°. For example, the inclination angle θ of the inclined surface 213 may be 8°, or the inclination angle θ of the inclined surface 213 may be 10°.

Preferably, the inclination angle θ of the inclined surface 213 is 5°.

Referring to FIG. 7, in order to make the condensed water generated in the airflow cavity 211 flow out more smoothly, the angle between a tangential direction of the arc surface 212 and a vertical direction ranges from 0° to 19°.

It should be noted here that the numerical values and numerical ranges involved in the present application are approximate values, and may have a certain range of error due to the influence of manufacturing process, and the error can be ignored by those skilled in the art.

In this embodiment, by arranging the arc surface 212, the condensed water in the airflow cavity 211 can flow away conveniently, which may prevent the condensed water from accumulating in the airflow cavity 211, thereby reducing accumulation of the condensed water in the nasal prong body 21, and reducing the potential risk that the patient is choked by the condensed water when breathing.

The structure of the contracted airflow cavity 211 conforms to the gas flowing law, will encounter a small airflow resistance, and is not easy to form condensed water. Moreover, the inclined surface 213 of the inner wall of one side of the airflow cavity 211 has a certain inclination angle, so that when the patient uses the oxygen inhalation nasal prong device, the whole nasal prong body 21 presents the inclination angle relative to the plane of the patient's nostril end, and thus the condensed water can smoothly flow back into the catheter assembly 10, avoiding the condensed water to accumulate and choke into the patient's nasal cavity, where the dotted line in FIG. 7 indicates the plane of the patient's nostril end.

Referring to FIGS. 4 and 5, the lanyard assembly 30 includes a first lanyard connecting member 31 and a second lanyard connecting member 34, which are respectively arranged at both sides of the nasal prong body 21. For example, as shown in FIG. 5, the first lanyard connecting member 31 is located at the upper side of the nasal prong body 21, and the second lanyard connecting member 34 is located at the lower side of the nasal prong body 21.

In one possible embodiment, the first lanyard connecting member 31 and the second lanyard connecting member 34 are integrally formed with the nasal prong body. The integrally formed structure enables that when wearing the oxygen inhalation nasal prong device, after the nasal prong body is placed at the patient's philtrum position, the first lanyard connecting member and the second lanyard connecting member will well matched with the curvature of the face, are more closely fit the human face and thus have small pressing feeling on the patient's face, which reduces the potential damage to skin of a patient with sensitive skin, and is comfortable to wear. The lanyard assembly 30 can well fix the nasal prong assembly 20, and will not cause compression damage to the patient's skin.

Of course, in another embodiment, the first lanyard connecting member 31 and the second lanyard connecting member 34 can also be connected with the nasal prong body by clamping, bonding or fastening.

The lanyard assembly 30 further includes a lanyard 32 connected between the first lanyard connecting member 31 and the second lanyard connecting member 34.

Specifically, the first lanyard connecting member 31 is connected with one end of the lanyard 32 through a connecting buckle 33, and the second lanyard connecting member 34 is connected with the other end of the lanyard 32 through another connecting buckle 33.

Specifically, the lanyard 32 is made of a soft and elastic material, such as rubber; the first lanyard connecting member 31 and the second lanyard connecting member 34 are made of a material with softness texture, good air permeability and little skin irritation, such as silicone rubber, so as to improve the comfort in use.

The lanyard 32 is provided with an adjusting buckle 35, and the lanyard 32 repeatedly passes through the adjusting buckle 35 to realize the function of adjusting the length of the lanyard 32, so as to be suitable for patients with different head circumference sizes.

The catheter assembly 10 includes an air conducting tube 11, an upper joint 13 and a lower joint 14, where the upper joint 13 and the lower joint 14 are respectively connected at both ends of the air conducting tube 11, and the nasal prong body 21 is through-connected with the upper joint 13.

Specifically, the air conducting tube 11 internally has an airflow channel 111, and the oxygen entering the airflow channel 111 is delivered to the airflow cavity 211 and then outputs from the air outlet 221.

The air conducting tube 11 is provided with a lanyard clamp 12, which is used for fixing the lanyard 32 onto the air conducting tube 11 so that when a patient wears the oxygen inhalation nasal prong device, the possibility of the nasal prong assembly 20 moving down or overturning due to gravity is reduced.

The air conducting tube 11 can be made of a plastic hose, and is retractable. It can also be straightened or curved, and can be changed and adjusted according to the needs of patients.

Preferably, the air conducting tube 11 may be a threaded tube.

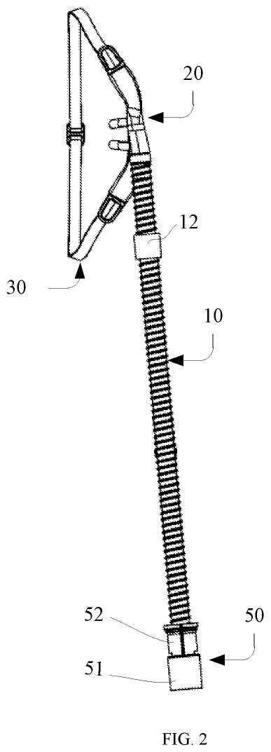

As another example of this embodiment, referring to FIG. 2, the oxygen inhalation nasal prong device further includes a joint assembly 50, the joint assembly 50 is through-connected to the other end of the catheter assembly 10, and the joint assembly 50 includes a machine joint 51 and a joint connecting member 52 connected to one end of the machine joint 51.

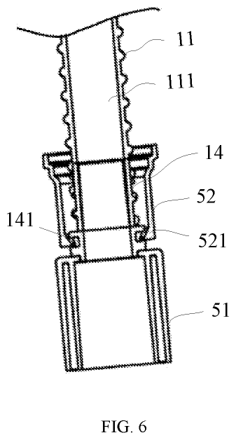

Referring to FIG. 6, the joint connecting member 52 is connected with the lower joint 14, and the machine joint 51 is configured to connect with an oxygen supply device or a ventilator-specific heated breathing pipeline.

A groove 141 is arranged on the periphery of the lower joint 14, and a claw 521 is arranged in the joint connecting member 52, and the claw 521 is matched with the groove 141, and the claw 521 is stuck in the groove 141, so that the joint connecting member 52 and the lower joint 14 can be snap-connected. Such a snap-connected mechanism is convenient to install, and allow the joint assembly 50 to freely rotate relative to the lower joint 14, so as to ensure that the air conducting tube 11 will not bend and twist during the period that the patient is wearing the oxygen inhalation nasal prong device.

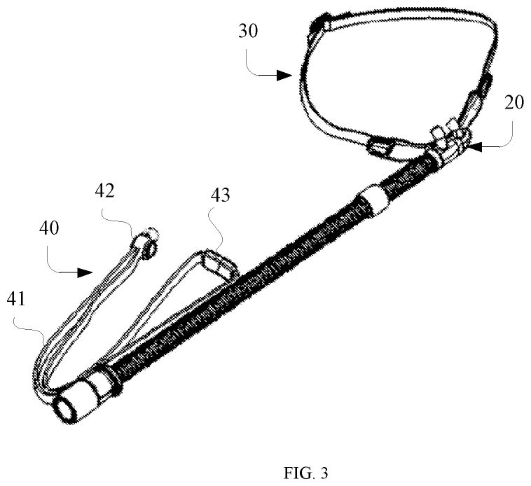

Referring to FIGS. 3 and 4, the neck strap assembly 40 includes a neck strap 41, a spring buckle 42 and a neck strap buckle 43, where two ends of the neck strap 41 are connected with each other through the neck strap buckle 43, and the spring buckle 42 is arranged on the neck strap 41. When the neck strap buckle 43 is buckled, the neck strap 41 becomes an annular whole, and when the neck strap buckle 43 is opened, the two ends of the neck strap 41 are separated. The neck strap 41 is configured to be worn around the neck of a patient and plays a role of fixing the air conducting tube 11. The neck strap buckle 43 can be easily opened and closed. Opening the neck strap buckle 43 allows the neck strap 41 to be easily tied around the patient's neck and then buckled, avoiding interference with the patient's head, neck and other parts, and facilitating medical staff to disassemble.

The spring buckle 42 passes through the neck strap 41, the spring buckle 42 is configured to adjust the length of the neck strap 41, and the length of the neck strap 41 may be adjusted according to the use requirements of different patients.

When in use, the machine joint 51 is connected to an oxygen supply device, and parameters of the oxygen supply device are set, for example, as follows: temperature is adjusted to 37° C., flow rate 40 L/min and a humidified gas flows out from the air outlet 221 of the nostril protruding pillar 22. The nasal prong body 21 is correctly worn on the patient's face, the nostril protruding pillar 22 is inserted into the patient's nostril, the first lanyard connecting member 31 and the second lanyard connecting member 34 are attached to the patient's face, the first lanyard connecting member 31 is connected with one end of the lanyard 32 through one connecting buckle 33, and then the second lanyard connecting member 34 is connected with another end of the lanyard 32 through another connecting buckle 33, and the length of the lanyard 32 is adjusted through the adjusting buckle 35, so that the lanyard assembly 30 exactly adapts to the size of the patient's head to ensure firm and comfortable wearing; the neck strap 41 is tied around the patient's neck, and the length of the neck strap 41 is adjusted to ensure firm and comfortable wearing. In this way, the oxygen inhalation nasal prong device is firmly fixed, avoiding the nasal prong assembly 20 to fall off due to the movement of the catheter assembly 10.

In the descriptions of the present application, it needs to be understood that, the terms used to indicate orientation or position relationships, such us “center”, “length”, “width”, “thickness”, “upper”, “lower”, “on”, “below”, “left” and “right”, “front” and “rear”, “vertical”, “horizontal”, “inner”, “outer”, “axial direction” and “circumferential direction” and the like are based on the orientation or position relationships shown in the appended drawings, and are only for the purpose of facilitating the descriptions of the present application and simplifying the descriptions, but not to indicate or imply that the referred orientation or members must have a particular orientation, specific construction and operation, and therefore they shall not be construed as a limitation of the present application.

In addition, the terms “first” and “second” are only used for descriptive purposes, and cannot be understood as indicating or implying relative importance or implicitly indicating the number of indicated technical features. Therefore, the features defined as “first” and “second” may include one or more of these features explicitly or implicitly. In the descriptions of the present application, “plural” means at least two, such as two, three, etc., unless otherwise specifically defined.

In the present application, unless otherwise specified and limited, the terms, for example, “install”, “connection”, “connect” and “fix” should be understood in a broad sense, such as they may be fixed connection, detachable connection or integrated connection; they may be mechanical connection, electrical connection or communicative connection; they may be direct connection, or indirect connection through an intermediate medium; they may be internal communication of two elements or interaction relationship between two elements. For those skilled in the art, the specific meanings of the above terms in the present application can be understood according to specific circumstances.

In the present application, unless otherwise specified and limited, a first feature on or under a second feature may include a situation in which the first feature is in a direct contact with the second feature, or may include a situation in which the first feature and the second feature are not in direct contact but are connected through another feature between them. Moreover, the first feature “above”, “on” and “over” the second feature includes situations in which the first feature is directly above and obliquely above the second feature, or only indicates that the horizontal height of the first feature is higher than the second feature. The first feature “lower”, “bellow” and “under” the second feature includes situations in which the first feature is directly below and obliquely below the second feature, or only indicates that the horizontal height of the first feature is smaller than the second feature.

Finally, it should be explained that the above embodiments are only used to illustrate the technical solutions of the present application, but not to limit it; although the present application has been described in detail with reference to the foregoing embodiments, it should be understood by those skilled in the art that the technical solutions described in the foregoing embodiments can still be modified, or some or all of the technical features therein can be equivalently replaced; however, these modifications or replacements do not make the essence of the corresponding technical solutions deviate from the scope of the technical solutions of the embodiments of the present application.

What is claimed is:

1. An oxygen inhalation nasal prong device, comprising:
a catheter assembly, configured as a hollow tubular structure;
a nasal prong assembly, arranged at one end of the catheter assembly, the other end of the catheter assembly being configured to communicate with an oxygen supply device, and the nasal prong assembly being connected with the catheter assembly in a through connection way;
a lanyard assembly, arranged on the nasal prong assembly, and the lanyard assembly being configured to be sleeved on a head of a patient, so that the nasal prong assembly is fixed at a nostril of the patient;
a neck strap assembly, configured to fix the other end of the catheter assembly to neck of the patient.

2. The oxygen inhalation nasal prong device according to claim 1, wherein the nasal prong assembly comprises a nasal prong body and a pair of nostril protruding pillars connected to a side of the nasal prong body, the nasal prong body is internally provided with a contracted airflow cavity, and the pair of nostril protruding pillars are each internally provided with an air outlet through-communicated with the airflow cavity.

3. The oxygen inhalation nasal prong device according to claim 2, wherein an inner wall of one side of the airflow cavity has an inclined surface, there is an inclination angle θ between the inclined surface and a plane of a nostril end of the patient, and an inner wall of the other side of the airflow cavity has an arc surface.

4. The oxygen inhalation nasal prong device according to claim 3, wherein the inclination angle θ remains unchanged along an inclined direction of the inclined surface relative to the plane.

5. The oxygen inhalation nasal prong device according to claim 3, the inclined surface is provided at one side of the airflow cavity close to the air outlet through-communicated with the airflow cavity, and the arc surface is provided at the other side of the airflow cavity far away from the air outlet through-communicated with the airflow cavity.

6. The oxygen inhalation nasal prong device according to claim 3, wherein the arc surface expands away from the inclined surface when it approaches an air inlet end of the airflow cavity; and the arc surface gradually approaches the inclined surface when it moves away from the air inlet end of the airflow cavity until it intersects with the inclined surface.

7. The oxygen inhalation nasal prong device according to claim 3, wherein the inclination angle θ of the inclined surface is 5°-15°.

8. The oxygen inhalation nasal prong device according to claim 7, wherein the inclination angle θ of the inclined surface is 5°.

9. The oxygen inhalation nasal prong device according to claim 2, wherein the lanyard assembly comprises a first lanyard connecting member and a second lanyard connecting member respectively arranged at both sides of the nasal prong body, and the first lanyard connecting member and the second lanyard connecting member are integrally formed with the nasal prong body;
the lanyard assembly further comprises a lanyard connected between the first lanyard connecting member and the second lanyard connecting member.

10. The oxygen inhalation nasal prong device according to claim 3, wherein the lanyard assembly comprises a first lanyard connecting member and a second lanyard connecting member respectively arranged at both sides of the nasal prong body, and the first lanyard connecting member and the second lanyard connecting member are integrally formed with the nasal prong body;
the lanyard assembly further comprises a lanyard connected between the first lanyard connecting member and the second lanyard connecting member.

11. The oxygen inhalation nasal prong device according to claim 4, wherein the lanyard assembly comprises a first lanyard connecting member and a second lanyard connecting member respectively arranged at both sides of the nasal prong body, and the first lanyard connecting member and the second lanyard connecting member are integrally formed with the nasal prong body;
the lanyard assembly further comprises a lanyard connected between the first lanyard connecting member and the second lanyard connecting member.

12. The oxygen inhalation nasal prong device according to claim 9, wherein the lanyard is provided with an adjusting buckle.

13. The oxygen inhalation nasal prong device according to claim 12, wherein the lanyard repeatedly passes through the adjusting buckle to adjust a length of the lanyard.

14. The oxygen inhalation nasal prong device according to claim 2, wherein the catheter assembly comprises an air conducting tube, an upper joint and a lower joint, the upper joint and the lower joint are respectively connected at both ends of the air conducting tube, and the nasal prong body is connected with the upper joint in a through connection way.

15. The oxygen inhalation nasal prong device according to claim 3, wherein the catheter assembly comprises an air conducting tube, an upper joint and a lower joint, the upper joint and the lower joint are respectively connected at both ends of the air conducting tube, and the nasal prong body is connected with the upper joint in a through connection way.

16. The oxygen inhalation nasal prong device according to claim 4, wherein the catheter assembly comprises an air conducting tube, an upper joint and a lower joint, the upper joint and the lower joint are respectively connected at both ends of the air conducting tube, and the nasal prong body is connected with the upper joint in a through connection way.

17. The oxygen inhalation nasal prong device according to claim 14, wherein the air conducting tube is provided with a lanyard clamp.

18. The oxygen inhalation nasal prong device according to claim 14, wherein the air conducting tube is a threaded tube.

19. The oxygen inhalation nasal prong device according to claim 14, further comprising a joint assembly, wherein the joint assembly is through-connected to the other end of the catheter assembly, the joint assembly comprises a machine joint and a joint connecting member connected to one end of the machine joint, and the joint connecting member is connected to the lower joint.
